(19) United States
(12) Patent Application Publication
(10) Pub. No.: US 2015/0088014 A1
(43) Pub. Date: Mar. 26, 2015

(54) PULSE DEPTH INDEX EXTRACTION DEVICE AND METHOD USING PRESSURE AT A CONSTANT VELOCITY

(71) Applicant: KOREA INSTITUTE OF ORIENTAL MEDICINE, Daejeon (KR)

(72) Inventors: Young Ju Jeon, Daejeon (KR); Jae Uk Kim, Daejeon (KR); Jang Han Bae, Daejeon (KR); Young Min Kim, Daejeon (KR); Jong Yeol Kim, Daejeon (KR); Si Woo Lee, Jeonju-si (KR)

(73) Assignee: KOREA INSTITUTE OF ORIENTAL MEDICINE, Daejeon (KR)

(21) Appl. No.: 14/399,930

(22) PCT Filed: May 8, 2013
(86) PCT No.: PCT/KR2013/004028

§ 371 Date: Nov. 7, 2014

(30) Foreign Application Priority Data
May 8, 2012 (KR) 10-2012-0048586

(51) Int. Cl. A61B 5/024

(52) U.S. Cl. CPC A61B 5/02444
USPC 600/500

(57) ABSTRACT

The present invention relates to a pulse depth index extraction device and method using pressure at a constant velocity. A device comprises: a pressure applying unit for applying a pressure at a constant velocity to a confirmed pulse location in a direction perpendicular to the skin; a pressure pulse wave signal measuring unit for measuring a pressure pulse wave signal generated based on the applied pressure; and a measured signal analysis unit for deriving the pulse depth index (PDI) by using the measured pressure pulse wave signal.

### TECHNICAL FIELD

The present invention relates to a method and apparatus for extracting a pulse depth index (PDI) based on a pressure at a constant velocity and, more particularly, to technology for determining a PDI based on a depth of a pulse location using a constant velocity pressurization scheme in a process of pulse diagnosis.

### BACKGROUND ART

In modern society, an interest in health consciousness is increasing continuously. With the interest of ages, corresponding technology is also being developed. For example, advancements are being made with a data analysis scheme and tool based on data collected in real time. Through this, it is possible to monitor an individual health condition and provide a personalized health management service.

Moreover, a change in consumer awareness may increase an expectation level and diversify needs of consumers. Accordingly, customization and facilitation for a health management service and related system may be improved, and a personalized health management program may rapidly grow based on a cumulative personal health database.

In previous years, the health management service provided to patients for healthcare was restricted to disease treatment in medical institutes and hospitals. In recent years, however, consumer needs for a health management program may be focused on advanced disease prevention and health maintenance.

Also, an improved standard of living may cause an increase in an interest in wellness. Thus, consumer preferences with respect to a preventive health management program such as a health condition measurement, an optimum amount of exercise, and the like may increase correspondingly.

Pulse wave features may be used for the health management program.

The pulse wave features may include at least one of a pulse wave measurement point, for example, Cun, Guan, and Chi, on left and right wrists of a user, a depth of a pulse location, a pulsation intensity, a pulse speed, a pulsation length, a pulsation width, an average pressure pulse wave in a pressurizing process of a pulse wave measurement, a pulse pressure, a diastole start height, an area of a pressure pulse wave per pulse pressure, a systolic pulse wave area, a pulse rate, a floating and deep condition index, a deficiency and excess condition index, a long and short condition index, a large and thread condition index, a slippery and hesitant condition index, a harmonic wave ratio, and an energy spectrum density function at a main harmonic frequency.

### DISCLOSURE OF INVENTION

### Technical Goals

An aspect of the present invention provides a method and apparatus for accurately estimate pulse depth information based on information on a period of time from a point in time of a sensor contacting a skin, to a point in time at which a predetermined component intensity of a maximum pressure pulse wave appears.

### Technical Solutions

According to an aspect of the present invention, there is provided a pulse depth index (PDI) determination apparatus including a pressure applying unit to apply a pressure at a constant velocity to a verified pulse location in a direction perpendicular to a skin, a pressure pulse wave signal measuring unit to measure a pressure pulse wave signal generated based on the applied pressure, and a measured signal analysis unit to derive a PDI using the measured pressure pulse wave signal.

The pressure applying unit may constantly apply the pressure at a velocity of 0.1 millimeters per second (mm/sec).

The pressure applying unit may determine a termination point in time of the pressure by verifying a point in time of a sensor contacting the skin, and a pattern in which the pressure pulse wave signal continuously increases based on the pressure constantly applied to the skin and starts to decrease at a predetermined point in time.

The pressure applying unit may suspend the applying of the pressure when a predetermined time for acquiring the pressure pulse wave signal using the pressure expires.

The measured signal analysis unit may derive the PDI based on information on a distance to which the skin is pressed by the sensor, by using information on a speed of a motor and information on a period of time from a point in time of a sensor contacting the skin, to a point in time corresponding to a predetermined component intensity of a maximum value of the pressure pulse wave signal, and the point in time corresponding to the predetermined component intensity may be, for example, a point in time of a maximum value, and a point in time corresponding to 50% or 20% of the maximum value.

According to another aspect of the present invention, there is also provided a PDI determination method including applying a pressure at a constant velocity to a verified pulse location in a direction perpendicular to a skin, measuring a pressure pulse wave signal generated based on the applied pressure, and deriving a PDI using the measured pressure pulse wave signal.

The applying may include determining a termination point in time of the pressure by verifying a point in time of a sensor contacting the skin, and a pattern in which the pressure pulse wave signal continuously increases based on the pressure constantly applied to the skin and starts to decrease at a predetermined point in time.

The applying may include suspending the applying of the pressure when a predetermined time for acquiring the pressure pulse wave signal using the pressure expires.

The deriving may include deriving the PDI based on information on a distance to which the skin is pressed by the sensor, by using information on a speed of a motor and information on a period of time from a point in time of a sensor contacting the skin, to a point in time corresponding to a predetermined component intensity of a maximum value of the pressure pulse wave signal, and the point in time corresponding to the predetermined component intensity may be, for example, a point in time of a maximum value, and a point in time corresponding to 50% or 20% of the maximum value.

### Advantageous Effects

According to an aspect of the present invention, it is possible to accurately estimate pulse depth information based on information on a period of time from a point in time of a sensor contacting a skin, to a point in time at which a predetermined component intensity of a maximum pressure pulse wave appears.

In contrast to an existing pulse wave or blood pressure measurement method using an applied pressure value as a reference measurement value, there is provided a constant velocity pressurization method using an application velocity as a reference measurement value in the present invention.

According to another aspect of the present invention, it is possible to provide a continuous pulse wave measurement method based on a constant velocity pressurization to directly acquire a travel distance of a sensor, thereby providing a higher accuracy for estimating a depth or a thickness of a vessel when compared to a multi-level pressurization method of a common pulsimeter.

### BRIEF DESCRIPTION OF DRAWINGS

FIG. 1 is a block diagram illustrating a pulse depth index (PDI) determination apparatus according to an example embodiment of the present invention.

FIG. 2 is a flowchart illustrating a PDI determination method according to an example embodiment of the present invention.

FIG. 3 is a flowchart illustrating an operation method of a pressure applying unit according to an example embodiment of the present invention.

FIG. 4A is a flowchart illustrating an operation method of a measured signal analysis unit according to an example embodiment of the present invention.

FIG. 4B is a diagram graphically illustrating an example embodiment of the operation method in FIG. 4A.

### BEST MODE FOR CARRYING OUT THE INVENTION

Hereinafter, example embodiments of the present invention will be described with reference to the drawings.

When it is determined detailed description related to a related known function or configuration they may make the purpose of the present invention unnecessarily ambiguous in describing the present invention, the detailed description will be omitted here. Also, terminologies used herein are defined to appropriately describe the exemplary embodiments of the present invention and thus may be changed depending on a user, the intent of an operator, or a custom. Accordingly, the terminologies must be defined based on the following overall description of this specification. Like reference numerals refer to like elements throughout.

As used herein, a pulse location-based pulse depth index (PDI) may refer to a pressurization distance to a point in time for acquiring a predetermined ratio, for example, 100%, 50%, and 20%, of a pressure pulse wave signal to a maximum pressure pulse wave signal.

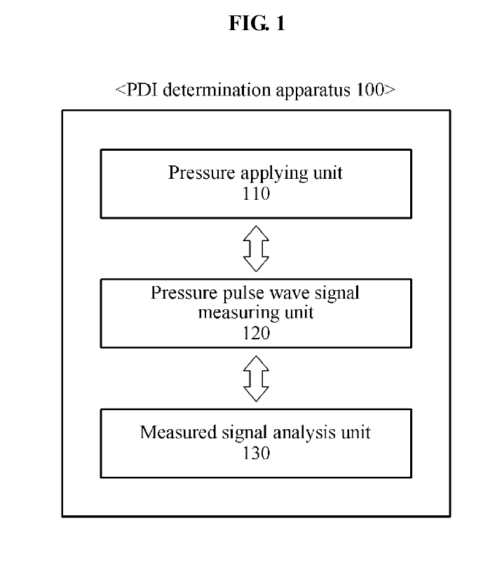

FIG. 1 is a block diagram illustrating a PDI determination apparatus 100 according to an example embodiment of the present invention.

The PDI determination apparatus 100 may include a pressure applying unit 110, a pressure pulse wave signal measuring unit 120, and a measured signal analysis unit 130.

The pressure applying unit 110 may apply a pressure at a constant velocity to a verified pulse location in a direction perpendicular to a skin.

The pressure pulse wave signal measuring unit 120 may measure a pressure pulse wave signal generated based on the applied pressure.

For example, the PDI determination apparatus 100 may apply the pressure at the constant velocity to the pulse location in the direction perpendicular to the skin and continuously measure the pressure pulse wave signal concurrently.

A velocity less than or equal to approximately 0.1 millimeters per second (mm/sec) may be used as a constant velocity of the pressure in an example of the present invention. Accordingly, the pressure applying unit 110 may apply the pressure at a velocity of 0.1 mm/sec.

The measured signal analysis unit 130 may derive the PDI using the measured pressure pulse wave signal.

The pressure applying unit 110 may suspend the applying of the pressure at an appropriate point in time.

The sensor may detach from the skin before the applying of the pressure is initiated.

As an example, the pressure applying unit 110 may determine a termination point in time of the pressure by verifying a point in time of the sensor contacting the skin and a pattern in which the pressure pulse wave signal increases based on the pressure constantly applied to the skin and decreases after a predetermined point in time.

As another example, the pressure applying unit 110 may suspend the applying of the pressure when a predetermined time period for acquiring the pressure pulse wave signal using the pressure expires.

The measured signal analysis unit 130 may derive the PDI based on information on a period of time from the point in time of the sensor contacting the skin, to a point in time corresponding to a predetermined component intensity of the pressure pulse wave signal.

According to an example embodiment of the present invention, pulse depth information may be accurately estimated based on information on a period of time from the point in time of the sensor contacting the skin, to a point in time at which the predetermined component intensity of a maximum pressure pulse wave signal appears.

In general, an applied pressure value may be used as a reference measurement value in existing pulse wave or blood pressure measurement methods. In the present invention, there is provided a constant velocity pressurization method using an application velocity as a reference measurement value.

According to the present invention, there is also provided a continuous pulse wave measurement method using the pressure to directly acquire a travel distance of the sensor. Thus, a higher accuracy may be achieved for estimating information on a thickness or a depth of a vessel when compared to a multi-level pressurization method of a common pulsimeter.

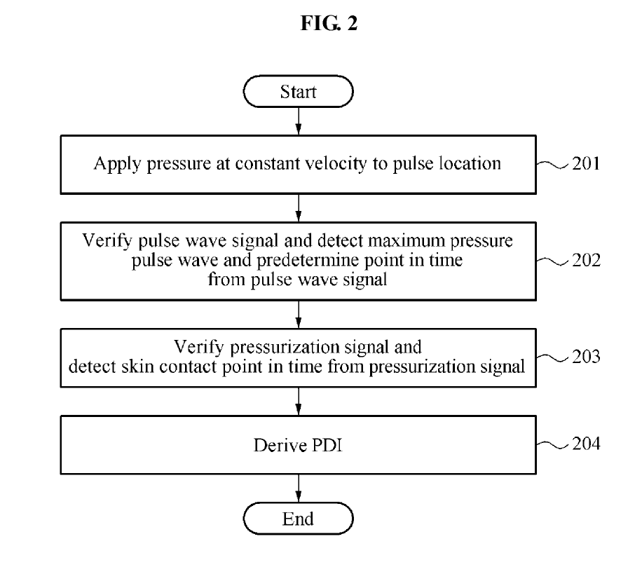

FIG. 2 is a flowchart illustrating a PDI determination method according to an example embodiment of the present invention.

The PDI determination method may apply a pressure at a constant velocity to a verified pulse location in a direction perpendicular to a skin using a pressure applying unit in operation 201, and may measure a pressure pulse wave signal generated based on the applied pressure.

Here, the PDI determination method may verify a pulse wave signal based on the applied pressure, and detect a maximum pressure pulse wave and a predetermined point in time from the verified pulse wave signal in operation 202.

The PDI determination method may verify a pressurization signal based on the applied pressure, and detect a skin contact point in time from the verified pressurization signal in operation 203.

According to an example embodiment of the present invention, operation 202 in which the maximum pressure pulse wave and the predetermined point in time are detected by verifying the pulse wave signal may be performed independently of operation 203 in which the skin contact point in time is detected by verifying the pressurization signal.

The PDI determination method may derive the PDI using a measured signal analysis unit based on at least one of the measured maximum pressure pulse wave signal, the predetermined point in time, and the skin contact point in time in operation 204.

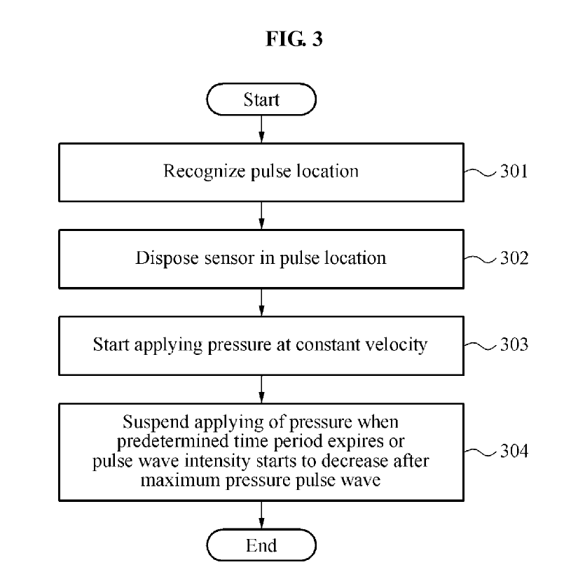

FIG. 3 is a flowchart illustrating an operation method of a pressure applying unit according to an example embodiment of the present invention.

The pressure applying unit may recognize a pulse location to apply a pressure at a constant velocity in operation 301.

The pressure applying unit may dispose a sensor in the recognized pulse location in operation 302, and may start applying the pressure at the constant velocity in operation 303.

In operation 304, the pressure applying unit may suspend the applying of the pressure when a predetermined time period expires or when a pulse wave intensity starts to decrease after a maximum pressure pulse wave.

Here, the pressure applying unit may determine a termination point in time of the pressure by verifying a point in time of a sensor contacting a skin, and a pattern in which the pressure pulse wave signal continuously increases based on the pressure constantly applied to the skin and decreases after a predetermined point in time.

Also, the pressure applying unit may suspend the applying of the pressure when the predetermined time period, for example, 120 seconds, for acquiring the pressure pulse wave signal using the pressure expires.

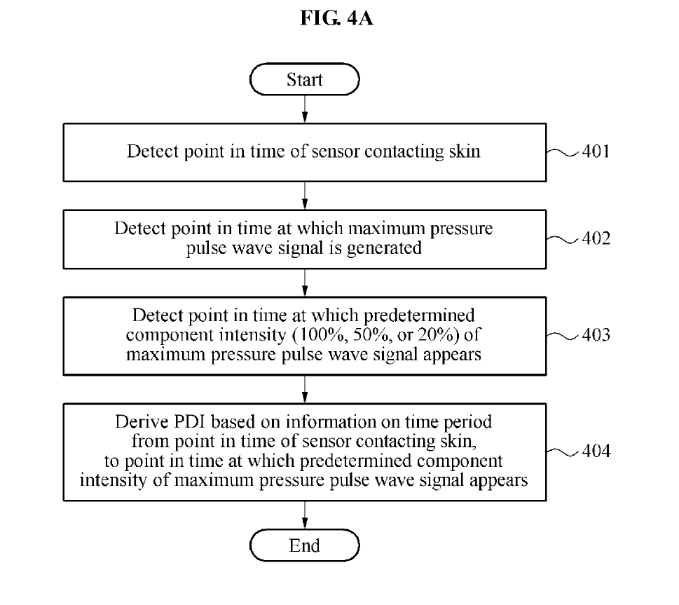

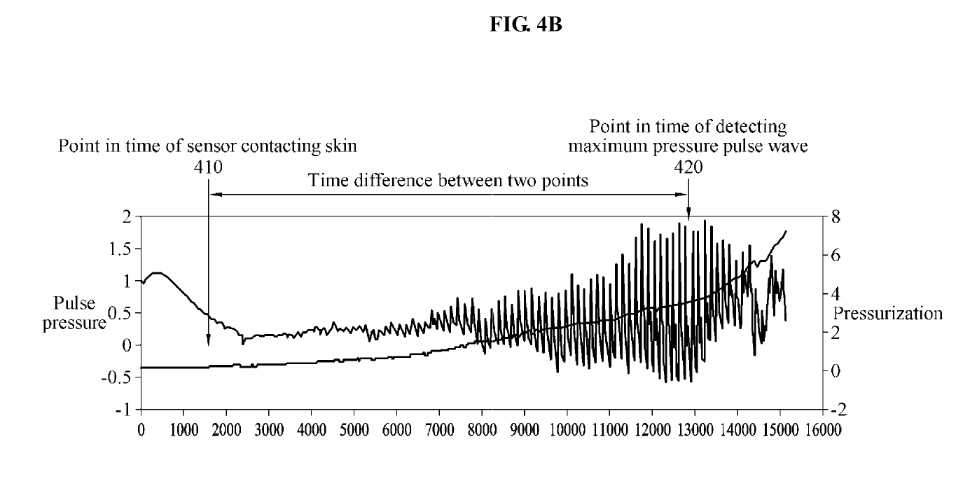

FIG. 4A is a flowchart illustrating an operation method of a measured signal analysis unit according to an example embodiment of the present invention.

The operation method of the measured signal analysis unit may detect a point in time of a sensor contacting a skin in operation 401, and detect a point in time at which a maximum pressure pulse wave signal is generated in operation 402.

The operation method of the measured signal analysis unit may detect a point in time at which a predetermined component intensity of the maximum pressure pulse wave signal appears in operation 403, and may derive a PDI based on information on a period of time from the point in time of the sensor contacting the skin, to the point in time at which the predetermined component intensity of the maximum pressure pulse wave signal appears in operation 404.

For example, the operation method of the measured signal analysis unit may detect a point in time at which at least one of intensities 100%, 50%, and 20% of the maximum pressure pulse wave appears.

FIG. 4B is a diagram graphically illustrating an example embodiment of the operation method in FIG. 4A.

The measured signal analysis unit may detect the PDI based on a point in time of a sensor contacting a skin as indicated by reference numeral 410 and a point in time of detecting a maximum pressure pulse wave as indicated by reference numeral 420.

For example, the measured signal analysis unit may detect the PDI by detecting a point in time 410 of the sensor contacting the skin and a point in time 420 at which the maximum pressure pulse wave is detected, and calculating a moving distance of the sensor based on a pressure applied at a constant velocity.

To calculate the moving distance of the sensor, the measured signal analysis unit may calculate a use time based on a time difference between the point in time 410 of the sensor contacting the skin and the point in time 420 at which the maximum pressure pulse wave is detected. The measured signal analysis unit may calculate the travel range of the sensor based on the calculated use time and a speed of a motor.

The PDI determination method according to the above-described embodiments may be recorded in non-transitory computer-readable media including program instructions to implement various operations embodied by a computer. The media may also include, alone or in combination with the program instructions, data files, data structures, and the like. Examples of non-transitory computer-readable media include magnetic media such as hard disks, floppy discs, and magnetic tape; optical media such as CD ROM discs and DVDs; magneto-optical media such as optical discs; and hardware devices that are specially configured to store and perform program instructions, such as read-only memory (ROM), random access memory (RAM), flash memory, and the like. Examples of program instructions include both machine code, such as produced by a compiler, and files containing higher level code that may be executed by the computer using an interpreter. The described hardware devices may be configured to act as one or more software modules in order to perform the operations of the above-described embodiments, or vice versa.

Concisely, by using the PDI determination method and apparatus according to an example embodiment of the present invention, it is possible to accurately estimate pulse depth information based on information on a period of time from a point in time of a sensor contacting a skin to a point in time at which a predetermined component intensity of a maximum pressure pulse wave appears.

While a few exemplary embodiments have been shown and described with reference to the accompanying drawings, it will be apparent to those skilled in the art that various modifications and variations can be made from the foregoing descriptions.

Thus, other implementations, alternative embodiments and equivalents to the claimed subject matter are construed as being within the appended claims.

1. A pulse depth index (PDI) determination apparatus comprising:
a pressure applying unit to apply a pressure at a constant velocity to a verified pulse location in a direction perpendicular to a skin;
a pressure pulse wave signal measuring unit to measure a pressure pulse wave signal generated based on the applied pressure; and
a measured signal analysis unit to derive a PDI using the measured pressure pulse wave signal.

2. The apparatus of claim 1, wherein the pressure applying unit determines a termination point in time of the pressure by verifying a point in time of a sensor contacting the skin, and a pattern in which the pressure pulse wave signal continuously increases based on the pressure constantly applied to the skin and starts to decrease at a predetermined point in time.

3. The apparatus of claim 1, wherein the pressure applying unit suspends the applying of the pressure when a predetermined time for acquiring the pressure pulse wave signal using the pressure expires.

4. The apparatus of claim 1, wherein the measured signal analysis unit derives the PDI based on information on a period of time from a point in time of a sensor contacting the skin, to a point in time corresponding to a predetermined component intensity of a maximum value of the pressure pulse wave signal.

5. A pulse depth index (PDI) determination method comprising:
applying a pressure at a constant velocity to a verified pulse location in a direction perpendicular to a skin;
measuring a pressure pulse wave signal generated based on the applied pressure; and
deriving a PDI using the measured pressure pulse wave signal.

6. The method of claim 5, wherein the applying comprises determining a termination point in time of the pressure by verifying a point in time of a sensor contacting the skin, and a pattern in which the pressure pulse wave signal continuously increases based on the pressure constantly applied to the skin and starts to decrease at a predetermined point in time.

7. The method of claim 5, wherein the applying comprises suspending the applying of the pressure when a predetermined time for acquiring the pressure pulse wave signal using the pressure expires.

8. The method of claim 5, wherein the deriving comprises deriving the PDI based on information on a period of time from a point in time of a sensor contacting the skin, to a point in time corresponding to a predetermined component intensity of a maximum value of the pressure pulse wave signal.

9. A non-transitory computer-readable storage medium comprising a program comprising instructions to cause a computer to perform the method of claim 5.

10. A non-transitory computer-readable storage medium comprising a program comprising instructions to cause a computer to perform the method of claim 6.

11. A non-transitory computer-readable storage medium comprising a program comprising instructions to cause a computer to perform the method of claim 7.

12. A non-transitory computer-readable storage medium comprising a program comprising instructions to cause a computer to perform the method of claim 8.
